(19) United States
(12) Patent Application Publication
(10) Pub. No.: US 2016/0324407 A1
(43) Pub. Date: Nov. 10, 2016

(54) TEST SYSTEM HAVING LASER TREATMENT FUNCTION

(71) Applicant: OPTIMEDE INC., Guro-gu Seoul (KR)

(72) Inventor: Hun Tae KIM, Seoul (KR)

(21) Appl. No.: 15/033,192

(22) PCT Filed: Oct. 31, 2014
(86) PCT No.: PCT/KR2014/010339

§ 371 Date: Jul. 14, 2016

(30) Foreign Application Priority Data
Oct. 31, 2013 (KR) 10-2013-0131131

(51) Int. Cl. A61B 1/06; A61B 18/22; A61B 1/00

(52) U.S. Cl. CPC A61B 1/0669; A61B 1/00096; A61B 1/063; A61B 18/22

(57) ABSTRACT

Provided is an inspection system having a laser treatment function, including: a first oscillator configured to generate a laser for image acquisition; a second oscillator configured to generate a laser for treatment; an irradiation device configured to irradiate the lasers generated from the first oscillator and the second oscillator to an inspection target, collect lasers reflected from the inspection target, and adjust a direction of a laser to be irradiated to the inspection target; a photo-sensor configured to receive the collected lasers and output an electrical signal; and a controller configured to visualize the output of the photo-sensor on a visualization device and control the first oscillator, the second oscillator, and the irradiation device in response to an input signal.

### TECHNICAL FIELD

The present disclosure relates to a system, and more particularly, to a test (inspection) system having a laser treatment function to treat a defect of an inspection target with a laser while inspecting the inspection target.

### BACKGROUND

Nowadays, with the rapid advancement of technology, inspection systems capable of inspecting whether there is a defect inside or outside an inspection target are being developed and applied to various industrial fields. For example, a medical endoscope system is being applied to a medical field in order to inspect whether there is a defect (or lesion) within organs of a human body, and an industrial endoscope system is being applied in order to inspect whether there is a defect within various kinds of industrial apparatuses.

Such an inspection system may have only an inspection function which is a basic function to inspect an inspection target and may further have a treatment function to treat a defect found while inspecting the inspection target. An inspection system having both the inspection function and the treatment function can perform an inspection and a treatment to an inspection target at the same time, which provides more convenience to an inspector or a therapist. Thus, the number of use cases has been on the increase.

As one example of an inspection system having both an inspection function and a treatment function, an endoscope system is disclosed in Korean Patent Laid-open Publication No. 10-2007-0074169. This endoscope system includes an endoscope to be inserted into a human body, and a camera, a lighting device, and a hyperthermia probe are provided at an end of the endoscope. The camera is configured to photograph the inside of the human body, and the lighting device is configured to project a light beam onto a site to be photographed by the camera. With the camera and the lighting device, the endoscope system disclosed in the above-described publication has the inspection function. Further, the hyperthermia probe is configured to generate heat to a predetermined temperature with a laser. With the hyperthermia probe, the endoscope system disclosed in the above-described publication also has the treatment function.

### DISCLOSURE OF THE INVENTION

### Problems to be Solved by the Invention

In the endoscope system disclosed in Korean Patent Laid-open Publication No. 10-2007-0074169, a configuration (camera, lighting device) for performing the inspection function and a configuration (hyperthermia probe) for performing the treatment function are completely separate. Therefore, the endoscope system disclosed in Korean Patent Laid-open Publication No. 10-2007-0074169 has a complicated structure.

The present disclosure is conceived to solve this problem. Thus, the present disclosure provides an inspection system having a laser treatment function, which has a simple structure and also performs both an inspection function and a treatment function to an inspection target.

### Means for Solving the Problems

According to an aspect of the present disclosure, there is provided an inspection system having a laser treatment function, including: a first oscillator configured to generate a laser for image acquisition; a second oscillator configured to generate a laser for treatment; an irradiation device configured to irradiate the lasers generated from the first oscillator and the second oscillator to an inspection target, collect lasers reflected from the inspection target, and adjust a direction of a laser to be irradiated to the inspection target; a photo-sensor configured to receive the lasers collected by the irradiation device and output an electrical signal; and a controller configured to visualize the output of the photo-sensor on a visualization device and control the first oscillator, the second oscillator, and the irradiation device in response to an input signal.

If the input signal is input to inspect the inspection target, the controller controls the irradiation device to adjust the direction of the laser to be irradiated to the inspection target according to a predetermined pattern and operates only the first oscillator of the first oscillator and the second oscillator.

If the input signal is input to treat the inspection target, the controller controls the irradiation device to adjust the direction of the laser to be irradiated to the inspection target according a predetermined pattern, and operates only the second oscillator at a treatment site included in the input signal and operates only the first oscillator in other sites.

The visualization device includes a touch screen, and desirably, the input signal may be input through the touch screen.

The inspection system having a laser treatment function may be an endoscope system. In this case, the first oscillator, the irradiation device, and the photo-sensor are provided within an endoscope.

### Effects of the Invention

According to the present disclosure, a means for irradiating and collecting a laser for image acquisition and a means for irradiating and collecting a laser for treatment are unified into one body. Therefore, the present disclosure has a simple structure and can be manufactured at low cost, as compared with the prior art.

Further, according to the present disclosure, a laser for treatment can be accurately irradiated to a site specified by an inspector. Therefore, a treatment can be performed with high accuracy.

Furthermore, according to the present disclosure, an input signal can be input simply by touching or dragging a touch screen.

Moreover, according to the present disclosure, there is provided an endoscope system from which all the above-described effects can be expected.

### BRIEF DESCRIPTION OF THE DRAWINGS

FIG. 1 is a system diagram illustrating an inspection system having a laser treatment function according to the present disclosure;

FIG. 2 is an enlarged view illustrating the inside of a portion A shown in FIG. 1;

FIG. 3 illustrates a screen of a visualization device of the inspection system illustrated in FIG. 1; and

FIG. 4 is a diagram provided to explain an example of a MEMS mirror.

### MODE FOR CARRYING OUT THE INVENTION

Hereinafter, embodiments of the present disclosure will be described in detail with reference to the accompanying drawings so that the present disclosure may be readily implemented by those skilled in the art. However, it is to be noted that the present disclosure is not limited to the embodiments but can be embodied in various other ways. In drawings, parts irrelevant to the description are omitted for the simplicity of explanation, and like reference numerals denote like parts through the whole document.

Through the whole document, the term “connected to” or “coupled to” that is used to designate a connection or coupling of one element to another element includes both a case that an element is “directly connected or coupled to” another element and a case that an element is “electronically connected or coupled to” another element via still another element. Further, through the whole document, the term “comprises or includes” and/or “comprising or including” used in the document means that one or more other components, steps, operation and/or existence or addition of elements are not excluded in addition to the described components, steps, operation and/or elements unless context dictates otherwise.

Terms and words used in the present specification and claims are not to be construed as a general or dictionary meaning, but are to be construed to meaning and concepts meeting the technical ideas of the present disclosure based on a principle that the inventors can appropriately define the concepts of terms in order to describe their own inventions in the best mode.

In the following, there will be described an example where an inspection system 100 having a laser treatment function according to the present disclosure is applied to a general medical endoscope including a head 10 and an insertion part 20. However, this example is provided only for clear understanding of the present disclosure, but does not mean that the present disclosure can be applied only to a general medical endoscope. The present disclosure can also be applied to other medical inspection systems or various kinds of industrial inspection systems.

The inspection system 100 according to the present disclosure includes a first oscillator 110, a second oscillator 120, an irradiation device 130, a photo-sensor (not illustrated), and a controller 150.

The first oscillator 110 may generate a first laser, and the second oscillator 120 may generate a second laser. Herein, the first laser and the second laser may be determined to be different from each other. To be more specific, at least one of a wavelength, a frequency, an output intensity, a type, a duration, or a speed of the first laser may be determined to be different from at least one of a wavelength, a frequency, an output intensity, a type, a duration, or a speed of the second laser. As one example, the first laser may be a visible laser and the second laser may be an infrared laser. As another example, the first laser may be an infrared laser and the second laser may be a visible laser. As yet another example, the first laser maybe an infrared laser and the second laser may be an X-ray laser. As still another example, the first laser may be a laser having a first wavelength selected from a range of 400 nm to 760 nm and the second laser may be a laser having a second wavelength selected from a range of 760 nm to 2000 nm. As still another example, the first laser may be a laser having a first wavelength selected from a range of 760 nm to 2000 nm and the second laser may be a laser having a second wavelength selected from a range of 400 nm to 760 nm. Further, types of the lasers may include, for example, a laser of infrared ray type, a laser of near-infrared ray type, a laser of red light type, a laser of blue light type, a laser of ultraviolet ray type, a laser of X-ray type, and a laser of ultrasonic wave type, or an ND-YAG laser having a wavelength of 1064 nm, a green laser having a wavelength of 532 nm, a hair removal laser having a wavelength of 808 nm, and a 15 W diode laser having a wavelength of 1470 nm.

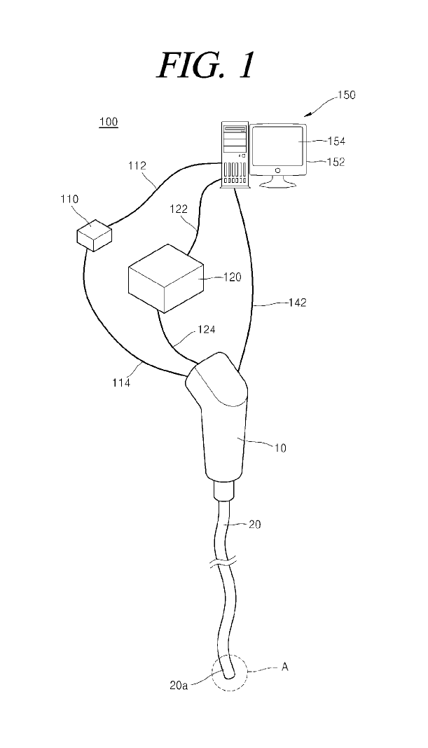

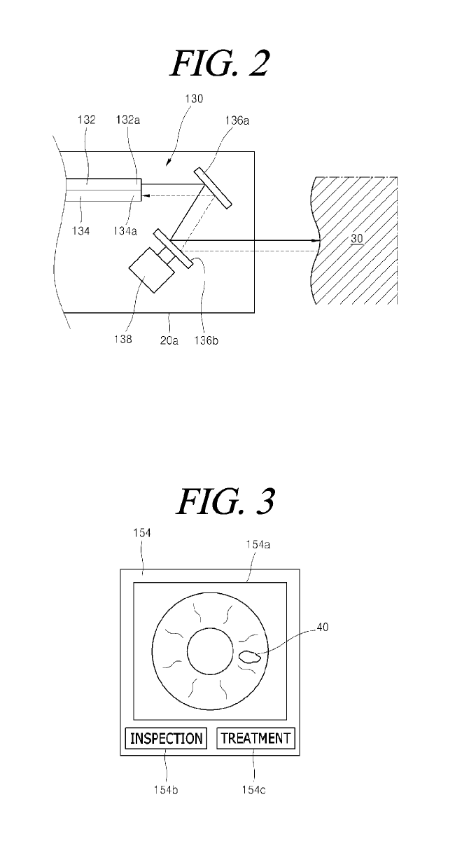

The first oscillator 110 may generate a laser for image acquisition, and the laser is used to acquire an image of an inspection target 30. The second oscillator 120 may generate a laser for treatment, and the laser is used to treat a lesion (or defect) present in the inspection target 30. The first oscillator 110 and the second oscillator 120 are connected to the controller 150 through electrical wires 112 and 122, respectively, and may be connected to the irradiation device 130 through optical fibers 114 and 124, respectively. Further, the first oscillator 110 and the second oscillator 120 may include a laser diode or an LED as a light source. The second oscillator 120 has a large size and thus cannot be provided within the head 10 of the endoscope. Therefore, the second oscillator 120 may be provided outside the head 10. Meanwhile, the first oscillator 110 has a relatively small size and thus can be provided within the head 10 of the endoscope, or may be provided outside the head 10. FIG. 1 illustrates an example where the first oscillator 110 is provided outside the head 10 of the endoscope.

The irradiation device 130 is configured to irradiate (or project) a laser, collect a laser, and adjust an irradiation direction of a laser. The irradiation device 130 may include a light-emitting fiber 132, a light-receiving fiber 134, a pair of mirrors 136a and 136b, and a MEMS (Micro Electro Mechanical System) 138.

The light-emitting fiber 132 may include a terminal end 132a and a front end (not illustrated) as the other end. The terminal end 132a of the light-emitting fiber 132 may be located within a terminal end 20a of the insertion part 20 of the endoscope. The light-emitting fiber 132 may be connected to at least one of the first oscillator 110, the second oscillator 120, the optical fiber 114 or the optical fiber 124, and may be detachably provided on at least one of them.

The front end of the light-emitting fiber 132 may be located within the head 10 of the endoscope, and may be connected to both the optical fiber 114 extended from the first oscillator 110 and the optical fiber 124 extended from the second oscillator 120.

The light-receiving fiber 134 may be provided in parallel with the light-emitting fiber 132, and may include a terminal end 134a and a front end (not illustrated) as the other end. The terminal end 134a of the light-receiving fiber 134 may be located within the terminal end 20a of the insertion part 20 of the endoscope and the front end thereof may be located within the head 10 of the endoscope. The light-receiving fiber 134 may be connected to a predetermined light-receiving module located within the photo-sensor (not illustrated) or the head 10, and may be detachably provided on any one of them.

Further, when the light-emitting fiber 132 and the light-receiving fiber 134 are detachably provided, sleeves may be provided at the ends of the light-emitting fiber 132 and the light-receiving fiber 134. A material of the sleeves may contain zirconia in order to suppress scratches or damage which may be generated during attachment and detachment.

The pair of mirrors 136a and 136b may guide a laser irradiated from the light-emitting fiber 132 to the inspection target 30 and guide a laser reflected from the inspection target 30 to the light-receiving fiber 134. The pair of mirrors 136a and 136b may include a fixed mirror 136a which cannot be moved and a MEMS mirror 136b combined with the MEMS 138. Herein, the MEMS 138 is a kind of driving means for adjusting an angle of the MEMS mirror 136b and may be controlled by the controller 150 while communicating with the controller 150. The mirrors 136a and 136b and the MEMS 138 may be located within the terminal end 20a of the endoscope. Meanwhile, the pair of mirrors 136a and 136b may be configured as MEMS mirrors. In this case, the MEMS 138 is provided as a pair and the pair of MEMSs 138 may be combined with the mirrors 136a and 136b, respectively. Further, the pair of mirrors 136a and 136b may be configured as fixed mirrors which cannot be moved. The irradiation device 130 is placed to include the above-described light-emitting fiber 132 and light-receiving fiber 134 therein to protect them, and may perform a function to adjust a position of a light to be irradiated to or reflected from the inspection target 30. The irradiation device 130 may include the fixed mirror 136a configured to reflect a light guided through the light-emitting fiber 132 toward a lower side and also reflect a light reflected from the inspection target 30 toward the light-receiving fiber 134.

Further, under the fixed mirror 136a, the MEMS 138 and the MEMS mirror 136b which can be rotated and adjusted in an inclination to a predetermined angle as being shaft-supported by the MEMS 138 may be positioned. The MEMS mirror 136b may perform a function to reflect a light reflected from the fixed mirror 136a toward the inspection target 30 and also perform a function to reflect a light reflected from the inspection target 30 toward the fixed mirror 136a while being rotated and adjusted in an inclination by the MEMS 138. However, the present disclosure is not limited thereto. The fixed mirror 136a may also be provided to be adjusted by the MEMS 138.

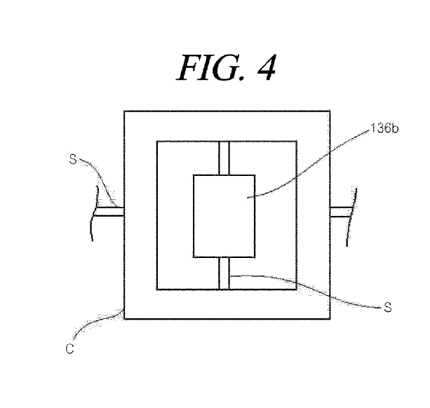

FIG. 4 is a diagram provided to explain an example of a MEMS mirror. According to the example with reference to FIG. 4, the MEMS mirror 136b is shaft S-supported by a case C or the like in order to adjust the MEMS mirror 136b. The case C may be shaft S-supported by a specific fixing means which is not illustrated herein.

The MEMS 138 for driving the MEMS mirror 136b may employ various devices, such as a micro motor configured to rotate the MEMS mirror 136b or a solenoid configured to adjust an angle of an adjusting mirror. The MEMS 138 may further include at least one of a piezoelectric ceramic and a piezoelectric film to be interlinked in response to a predetermined signal. The MEMS 138 may further include at least one of an electrostatic MEMS driving device, a magnetic MEMS driving device or a piezoelectric MEMS driving device to be interlinked in response to a predetermined signal.

Meanwhile, a collimator 260 as a light-receiving lens for improving the straightness of a light and the light-concentrating property may be located on a light path between the fixed mirror 136a and the light-emitting mirror 132 and between the fixed mirror 136a and the light-receiving fiber 134. However, the present disclosure is not limited thereto. In addition to the collimator 260, multiple lenses may be further provided to improve the straightness and the light-concentrating property.

Hereinafter, the irradiation device 130 according to another exemplary embodiment will be described. In this case, it is assumed that the fixed mirror 136a serves as another MEMS mirror 136a. The MEMS mirror 136a vibrates at a predetermined angle in one of an X-axis direction or a Y-axis direction, and the MEMS mirror 136b vibrates in the other one direction. Thus, the MEMS mirrors 136a and 136b may irradiate a light to the inspection target 30 or receive a light received from the inspection target 30 while moving along the X-axis and Y-axis directions. Since the vibration is made along the two axes as such, a light can be received from an area having a predetermined size and an image can be produced. The light may be a continuous light or a pulse light.

A 2D optic micro electronic mechanical system (MEMS) or 1D MEMS to be interlinked in response to a predetermined signal may be used as the MEMS 138. Herein, 2D means driving in both X-axis and Y-axis directions and 1D means driving in one direction. The MEMS 138 may be any one of the above-described solenoid, micro motor, piezoelectric film, piezoelectric element, and MEMS, or may be a combination formed by selecting and combining some of them.

The photo-sensor (not illustrated) may be located within the head 10 of the endoscope and maybe connected to the front end of the light-receiving fiber 134. Further, the photo-sensor may be connected to the controller 150 through an electrical wire 142. The photo-sensor may receive a laser from the light-receiving fiber 134 and then converts and outputs the laser into an electrical signal to the controller 150. Examples of the photo-sensor may include an image sensor or a photo-diode.

Hereinafter, an exemplary embodiment of the present disclosure will be described with reference to the above-described configuration. If a light is irradiated from a laser diode or an LED serving as the first oscillator 110, the light is guided by the light-emitting fiber 132 and irradiated to the inspection target 30 through the fixed mirror 136a located adjacent to the terminal end and the MEMS mirror 136b located under the fixed mirror 136a. In this case, any one or each of the fixed mirror 136a and the MEMS mirror 136b may guide the light to an accurate inspection site while being interlinked by the MEMS 138. Further, a light reflected from the inspection target 30 is guided to an image sensor or a photo-diode by the light-receiving fiber 134 through the MEMS mirror 136b and the fixed mirror 136a in reverse order, and an output of the image sensor or the photo-diode is transmitted to a monitor M through the controller 150. Thus, an inspector can check a video or photo of the inspection site with the naked eye.

Hereinafter, another exemplary embodiment of the present disclosure will be described with reference to the above-described configuration. If a light is irradiated from a laser diode or an LED serving as the second oscillator 120, the light is guided by the light-emitting fiber 132 and irradiated to the inspection target 30 through the fixed mirror 136a located adjacent to the terminal end and the MEMS mirror 136b located under the fixed mirror 136a. The irradiated light may treat or relieve a defect (or lesion) within the inspection target 30. In this case, any one or each of the fixed mirror 136a and the MEMS mirror 136b may guide the light to an accurate inspection site while being interlinked by the MEMS 138.

Hereinafter, yet another exemplary embodiment of the present disclosure will be described with reference to the above-described configuration. A laser for image acquisition generated from the first oscillator 110 may be irradiated to the inspection target 30 through the optical fiber 114, the light-emitting fiber 132, and the pair of mirrors 136a and 136b in sequence and then reflected from the inspection target 30. Then, the reflected laser may be collected by the photo-sensor (not illustrated) through the pair of mirrors 136a and 136b and the light-receiving fiber 134.

In this case, the laser for image acquisition may be irradiated to a site of interest in the inspection target along a specific irradiation line (e.g., an X-axis irradiation line or a Y-axis irradiation line). Then, the photo-sensor converts and outputs the laser for image acquisition into an electrical signal. The controller 150 may produce an image with the electrical signal. Meanwhile, while visualizing the site of interest in the inspection target along the irradiation line, the controller 150 may recognize that it is necessary to visualize a site corresponding to a defect (or lesion) within the inspection target and control the second oscillator 120 to irradiate a laser for treatment to the site corresponding to the defect (or lesion). In this case, the laser for treatment may be irradiated to the inspection target 30 through the light-emitting fiber 132 and the pair of mirrors 136a and 136b in sequence and thus may be used to treat or relieve the defect (or lesion). In this case, irradiation of the laser for image acquisition may be stopped while the laser for treatment is irradiated. Then, the controller 150 may stop irradiation of the laser for treatment and restart irradiation of the laser for image acquisition. In this case, the controller 150 may irradiate the laser for treatment for a specific duration and then restart irradiation of the laser for image acquisition, or may recognize that an irradiation location on the irradiation line within the inspection target is not present in the site corresponding to the defect (or lesion) and then restart irradiation of the laser for image acquisition along the irradiation line.

In other words, if a defect (or lesion) is found from an image acquired by irradiating the laser for image acquisition, the controller 150 may recognize and/or display a location of the defect and transmit an instruction or signal to irradiate the laser for treatment to the second oscillator 120 such that the laser for treatment can be irradiated to the location of the defect. The second oscillator 120 may irradiate the laser for treatment to the location of the defect included in a trajectory of the irradiated laser for image acquisition, on the basis of the instruction or signal to irradiate the laser for treatment. Further, the laser for treatment is not irradiated to other sites except the location of the defect, but the laser for image acquisition may be irradiated thereto by the first oscillator 110. According to an exemplary embodiment of the present disclosure, while the laser for treatment is irradiated by the second oscillator 120, the laser for image acquisition may be irradiated by the first oscillator 110. Otherwise, according to another exemplary embodiment of the present disclosure, while the laser for treatment is irradiated by the second oscillator 120, the first oscillator 110 may stop irradiation of the laser for image acquisition to protect a light-receiving element from a powerful light (laser).

According to an exemplary embodiment of the present disclosure, the light-emitting fiber 132 and the light-receiving fiber 134 may be formed as one fiber. In this case, a laser output from the first oscillator 110 or the second oscillator 120 may be irradiated to the fixed mirror 136a through the fiber, and a light (or ray, laser) reflected from the inspection target 30 may be guided to the image sensor or the photo-diode through the MEMS mirror 136b, the fixed mirror 136a, a terminal end of the fiber, and the fiber.

The controller 150 includes a visualization device such as a monitor and is loaded with a predetermined driving program, and may have an operational function to install and execute the driving program. A desktop computer, a notebook computer, a smart phone, a smart pad, and other kinds of computers may be used as the controller 150. Hereinafter, an operational process of the controller 150 after execution of the driving program will be described in detail.

If the driving program is executed, the controller 150 assigns an image sector 154a on a screen 154 of the visualization device 152 and then displays the image sector 154a together with an inspection icon 154b and a treatment icon 154c on the screen 154.

Then, if the inspector clicks the inspection icon 154b, the controller 150 may operate the first oscillator 110. Then, a laser for image acquisition generated from the first oscillator 110 is irradiated to the inspection target 30 through the optical fiber 114, the light-emitting fiber 132, and the pair of mirrors 136a and 136b in sequence and then reflected from the inspection target 30. Then, the reflected laser may be collected by the photo-sensor (not illustrated) through the pair of mirrors 136a and 136b and the light-receiving fiber 134. Then, the photo-sensor converts and outputs the laser for image acquisition into an electrical signal. The controller 150 may visualize the electrical signal on the image sector 154a.

If the MEMS 138 does not operate during the above-described process, since the laser for image acquisition is irradiated only to one site in the inspection target 30, any point rather than a predetermined site may be visualized on the image sector 154a. Therefore, if the inspector clicks the inspection icon 154b, the controller 150 may operate the MEMS 138 according to a predetermined pattern such the predetermined site can be visualized on the image sector 154a. The pattern may be determined in various ways. For example, the pattern may be determined in such a way that a laser for image acquisition moves from left to right and slightly moves down, moves from right to left and slightly moves down, and then moves from left to right.

If a lesion (or defect) 40 is detected from an inspection image while the above-described inspection process is performed, the inspector may specify the lesion 40. Such designation may be made by inputting coordinates of a site for the lesion 40 through a keyboard or displaying the site for the lesion 40 on the image sector 154a through a mouse. Then, if the inspector clicks the treatment icon 154c, the controller 150 selectively operates the first oscillator 110 and the second oscillator 120 while operating the MEMS 138 according to the above-described pattern. In this case, the controller 150 operates only the first oscillator 110 at a site which is not specified by the inspector, and, thus, a laser for image acquisition is irradiated to the inspection target 30. Further, the controller 150 operates only the second oscillator 120 at a site which is specified by the inspector, and, thus, a laser for treatment is irradiated to the inspection target 30. In this case, both the laser for image acquisition and the image for treatment are visualized through the above-described process. Thus, a process of treating the lesion 40 can be visualized on the image sector 154a.

If the treatment for the lesion 40 is completed, the inspector may click the inspection icon 154b again to continue the inspection.

Meanwhile, the visualization device 152 may include a touch screen. In this case, the inspector can easily input an instruction by touching the inspection icon 154b or the treatment icon 154c displayed on the touch screen. If the visualization device 152 includes the touch screen, the controller 150 may receive coordinates of a treatment site through a drag (scratching the touch screen being touched). In this case, the inspector can easily input the coordinates of the treatment site.

The touch screen may be of any one of electrostatic type and pressure type. However, an endoscopy operator usually wears sanitary gloves. Thus, if the touch screen 152 is of electrostatic type, he/she may need to use a touch pen or wear electrostatic touch gloves as sanitary gloves.

The above description of the present disclosure is provided for the purpose of illustration, and it would be understood by those skilled in the art that various changes and modifications may be made without changing technical conception and essential features of the present disclosure. Thus, it is clear that the above-described embodiments are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

1. An inspection system having a laser treatment function, comprising:
a first oscillator configured to generate a laser for image acquisition;
a second oscillator configured to generate a laser for treatment;
an irradiation device configured to irradiate the lasers generated from the first oscillator and the second oscillator to an inspection target, collect lasers reflected from the inspection target, and adjust a direction of a laser to be irradiated to the inspection target;
a photo-sensor configured to receive the collected lasers and output an electrical signal; and
a controller configured to visualize the output of the photo-sensor on a visualization device and control the first oscillator, the second oscillator, and the irradiation device in response to an input signal.

2. The inspection system having a laser treatment function of claim 1,
wherein if the input signal is input to inspect the inspection target, the controller controls the irradiation device to adjust the direction of the laser to be irradiated to the inspection target according to a predetermined pattern and operates only the first oscillator of the first oscillator and the second oscillator.

3. The inspection system having a laser treatment function of claim 1,
wherein if the input signal is input to treat the inspection target, the controller controls the irradiation device to adjust the direction of the laser to be irradiated to the inspection target according a predetermined pattern, and operates only the second oscillator at a treatment site included in the input signal and operates only the first oscillator in other sites.

4. The inspection system having a laser treatment function of claim 1,
wherein the visualization device includes a touch screen, and the input signal is input through the touch screen.

5. The inspection system having a laser treatment function of claim 1,
wherein the first oscillator, the irradiation device, and the photo-sensor are provided within an endoscope.
